(19) United States
(12) Patent Application Publication
(10) Pub. No.: US 2002/0065548 A1
(43) Pub. Date: May 30, 2002

(54) Profiled stent and method of manufacture

(76) Inventors: Matthew J. Birdsall, Santa Rosa, CA (US); Robert D. Lashinski, Sebastopol, CA (US); Randall T. Lashinski, Santa Rosa, CA (US)

Correspondence Address: MEDTRONIC AVE, INC., 3576 UNOCAL PLACE, SANTA ROSA, CA, 95403, US

(21) Appl. No.: 10/029,553

(22) Filed: Dec. 20, 2001

Related U.S. Application Data
(63) Continuation of application No. 09/608,882, filed on Jun. 30, 2000, now abandoned.
Application No. 09/608,882 is a continuation of application No. 08/702,258, filed on Aug. 23, 1996, now abandoned.

(51) Int. Cl. A61F 2/06

(52) U.S. Cl. 623/1.15

(57) ABSTRACT

A profiled stent for supporting and maintaining the patency of lumens in living tissue. The profiled stent includes a plurality of support members, each of the support members having a first side, a second side positioned opposite the first side, a third side adjoining the first and second sides, a fourth side positioned opposite the third side adjoining the first and second sides. Four corners are defined by the adjoining first, second, third, and fourth sides. At least one of the support members of the stent is profiled such that the four corners of that support member are round, while the first and second sides are substantially flat or, stated differently, exhibit substantially similar radii of curvature.

This application is a continuation of application Ser. No. 08/702,258, filed Aug. 23, 1996.

### FIELD OF THE INVENTION

This present invention relates to intravascular stents for maintaining the patency of lumens in living tissue. And, more specifically, to a profiled stent and method of manufacture therefor.

### BACKGROUND OF THE INVENTION

Percutaneous transluminal coronary angioplasty (“PTCA”) is a now common procedure for treating coronary artery disease. PTCA typically involves advancing a catheter, having an inflatable balloon on the distal end thereof, through a patient's arterial system until the balloon crosses an atherosclerotic lesion. The balloon is then inflated to dilate the artery. After dilation, the balloon is deflated and the catheter removed leaving an enlarged arterial passageway or lumen, thereby increasing blood flow. A significant number of PTCA procedures, however, result in a restenosis or renarrowing of the lumen.

To lessen the risk of stenosis or restenosis of lumens, various endoprosthetic devices have been proposed for mechanically keeping an affected lumen open after completion of procedures, such as PTCA. For purposes of the instant invention, a lumen can be a blood vessel, a bile duct, or any other similar body conduit that tends to improperly constrict as a result of disease or malfunction. A lumen may also be a graft (whether natural or artificial) in any type of body conduit.

Endoprosthetic devices generally referred to as stents, are typically inserted into the lumen, positioned across a lesion, and then expanded to keep the passageway clear. Effectively, the stent overcomes the natural tendency of some lumen walls to close due to restenosis, thereby maintaining a more normal flow of blood through that lumen than would be possible if the stent were not in place or if only a PTCA procedure were performed.

There are two general categories of stents, self-expanding stents and balloon-expandable stents. Some self-expanding stents are made from a tube of stainless wire braid. Such stents are typically compressed into a first shape and inserted into a sheath or cartridge. During insertion, the stent is positioned along a delivery device, such as a catheter, that is extended to make the stent diameter as small as possible. When the stent is positioned across the lesion, the sheath is withdrawn causing the stent to radially expand and abut the vessel wall. Depending on the materials used in construction of the stent, the tube maintains the new shape either through mechanical force or otherwise.

The stent is then delivered to the affected area on a catheter. Once properly positioned, the stent is allowed to expand.

Another type of self-expanding stent is made from a shape-memory alloy such as NITINOL. This stent has been pre-treated to assume an expanded state at body temperature. Prior to delivery to the affected area, the stent is typically crimped or compressed near or below at room temperature.

Balloon-expandable stents are typically introduced into a lumen on a catheter having an inflatable balloon on the distal end thereof. When the stent is at the desired location in the lumen, the balloon is inflated to circumferentially expand the stent. The balloon is then deflated and the catheter is withdrawn, leaving the circumferentially expanded stent in the lumen, usually as a permanent prosthesis for helping to hold the lumen open.

One type of balloon-expandable stent is a tubular-slotted stent, which involves what may be thought of as a cylinder having a number of slots cut in its cylindrical wall, resulting in a mesh when expanded. A tubular-slotted stent is cut out of a tube, typically a hypo-tube, or out of a sheet, which is then rolled, and then welded to form a cylinder. Tubular-slotted stents that are cut out of a tube typically have a rectangular cross-section, which produces rather sharp and square edges that remain even after polishing. As a result, such tubular-slotted stents may have a tendency to dissect the lumen as the stent is advanced through the lumen on the catheter.

A balloon-expandable stent referred to as a wire stent overcomes some of the problems associated with tubular-slotted stents. A wire stent is generally formed by winding a circular shaped wire into supportive elements, which typically have a circular cross-section. The problem with wire stents is that the supportive elements comprising the stent can axially displace with respect to each other, resulting in a stent that fails to provide adequate support.

U.S. Pat. No. 5,292,331 issued to Boneau, which is hereby incorporated by reference discloses another type of wire stent, referred to here as a Boneau stent. A Boneau stent is made by taking a ring or toroid having a circular cross-section, and then forming the ring into a series of sinusoidally-shaped elements. While preferably employing a single piece of material, suitably welded wire, is also acceptable. A Boneau stent bridges the gap between tubular-slotted stents and wire stents by retaining the flexibility of wire stents, while approaching the axial stability of tubular-slotted stents.

While conventional stents have been found to work well, conventional stents suffer from several disadvantages. As stated above, stents that have a rectangular cross-section may damage the inner walls of a lumen due to sharp edges. And stents having a rounded cross-section, while reducing the risk of dissection or trauma, neither possess an efficient surface-to-wall covering ratio nor efficient strength for material volume.

Accordingly, what is needed is an improved stent structure that makes efficient use of stent material while reducing the risk of trauma to the lumen wall. The present invention addresses such a need.

### SUMMARY OF THE INVENTION

The present invention provides a profiled stent for helping to hold open a lumen. The profiled stent comprises at least one support member having at least a first side, a second side, and a third side, and three rounded edges defined where the first second and third sides meet.

According to the apparatus and method disclosed herein, the present invention increases the radial strength of the stent and increases the efficiency of surface coverage. Furthermore, rounded edges are retained on the stent, which provides less traumatic trackability as the stent is advanced through a lumen.

Therefore, it is an object of the instant invention to provide a stent with increased load-carrying capability.

It is a further object of the invention to provide a stent which optimizes the stent surface to lumen wall coverage.

It is also an object of the invention to displace stent material to higher stressed regions.

These and other advantages are realized while retaining rounded edges on the stent so that it remains less traumatic.

### BRIEF DESCRIPTION OF THE DRAWINGS

FIG. 1 is a side view of an illustrative embodiment of a stent used for forming a profiled stent embodying the principles of the present invention.

FIGS. 2a-2c are cross-section views of sections or various stents.

FIGS. 3a and 3b are cross-section views of a profiled section of a stent in accordance with the present invention.

FIG. 4 is an isometric view of the Boneau stent shown in FIG. 1 that has undergone the process of profiling in accordance with the present invention to produce a profiled stent.

FIG. 5 is an end view of a six crown non-profiled Boneau stent.

FIG. 6 is an end view of a six crown profiled Boneau stent.

FIG. 7 is a flow chart depicting the process of producing a profiled stent in accordance with the present invention.

FIG. 8 is a block diagram showing the profiling of a stent using a rotary swaging machine.

FIG. 9 is a block diagram showing the profiling of a stent using a collet.

FIG. 10 is a block diagram showing the profiling of a stent using a roller machine.

FIG. 11 is a block diagram showing the profiling of a stent using a sizing tube and forming tool.

### DETAILED DESCRIPTION OF THE PREFERRED EMBODIMENT

The present invention relates to a profiled stent and a method of manufacture therefor. The following description is presented to enable one of ordinary skill in the art to make and use the invention and is provided in the context of a patent application and its requirements. Various modifications to the preferred embodiment will be readily apparent to those skilled in the art and the generic principles herein may be applied to other embodiments. Thus, the present invention is not intended to be limited to the embodiment shown but is to be accorded the widest scope consistent with the principles and features described herein.

The present invention provides a profiled stent formed from a stent of conventional design. Although stents may be constructed in many different ways, the profiling method of the present invention is applicable to all known stent constructions, and it will be readily apparent from the following discussion of several exemplary constructions how the invention can be applied to any other type of stent construction.

FIG. 1 is a side view of an illustrative embodiment of a stent used for forming a profiled stent embodying the principles of the present invention. As described in the Boneau patent, an illustrative stent 10 includes five sections 12a-e, each of which is made of an endless metal loop that has been bent into a plurality of straight sections or struts 13 that are integrally joined by discrete axial turns, or crowns 14. Each section 12 may have more undulations than are shown in FIG. 1, but the simplified depictions shown herein will be sufficient to illustrate the present invention.

Although sections 12 may or may not be made of what would be regarded in some other arts as wire, the material of sections 12 is generally wire-like, and so the term “wire” is sometimes used herein to refer to such stent material. Axially adjacent sections 12 may be joined to one another at one or more of their crowns 14. These connections (if and to the extent present) may be made by welding, soldering, adhesive bonding, mechanical fastening, or in any other suitable manner.

A typical technique for delivering stents of the general type shown in FIG. 1 into a lumen is to initially dispose of the stent structure in a circumferentially compressed form around a deflated balloon which is part of a balloon catheter. The catheter is then inserted axially into a tubular body structure to be stented until the balloon and stent are at the desired location along the body structure. The balloon is then inflated to circumferentially expand the stent. Lastly, the balloon is deflated and the catheter is withdrawn, leaving the expanded stent behind in the body structure.

The deformation of the stent produced by the balloon as described above is at least partly permanent. As used here, such permanent deformation will be referred to as “plastic”. It will be understood that the terms “plastic”, “plastically”, or the like as used herein mean any type of non-elastic or permanent deformation, whether in the traditional materials science sense, and therefore, due to straining some portion of the stent material beyond its elastic limit, or as a result of any other property of the stent material or structure which results in the deformed stent taking a “set”, which is different from its initial set. Correspondingly, the term “yield strength” means the point at which the stent structure or its material transitions from elastic to plastic deformation, as the term “plastic” is broadly defined above.

The balloon is strong enough to overcome the yield strength of the stent, but when the balloon is no longer radially supporting the stent, the surrounding tubular body structure does not exert sufficient radially inward force on the stent to overcome the stent's yield point to the extent that the stent returns to its original diameter.

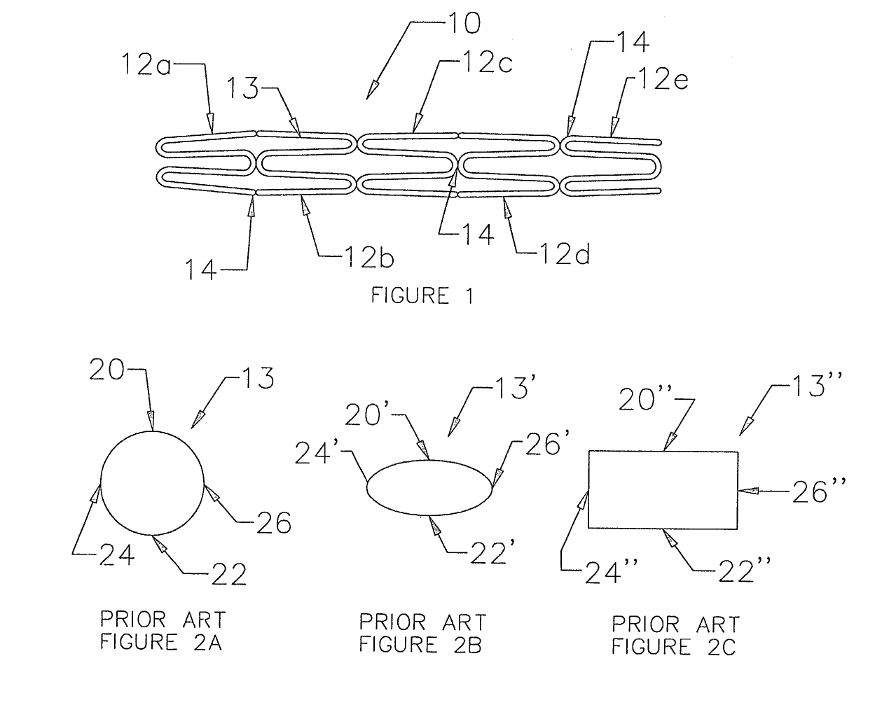

FIGS. 2a-2c are cross-section views of struts or support members 13 of various stent sections. FIG. 2a is a cross-section view of stent 10 shown in FIG. 1, which has a circular cross-sectional shape. Or as shown FIG. 2b, the cross-sectional shape of strut 13′ may be ellipsoidal. As stated above, stents may be constructed in many different ways. One feature common to conventional stents, however, is that they all include some type of support member or members that have substantially the same cross-sectional dimensions. Referring to FIG. 2c, besides circular and ellipsoidal cross-sectional shapes, a stent section 12″ may have a rectangular cross-sectional shape as shown, or a hexagon, square, or other geometric shape.

Referring to FIGS. 2a-2c, the cross sections of all types of support members may be described as generally defined by a top portion 20,20′,20″ a bottom portion 22,22′22″ side portion 24,24′,24″, and an opposing side portion 26, 26′,26″. As shown in FIGS. 2a-2c, once the stent is disposed and then expanded inside a lumen, the top portion 20,20′,20″ of a support member is the part that abuts against and supports the wall of the lumen. Therefore, the top and bottom portions 20,20′,20″ and 22,22′,22″ are stressed more than the side portions 24, 24′,24″ and 26, 26′,26″ of the support member.

Stents having traditionally shaped cross-sections as shown in FIGS. 2a-2c suffer from various disadvantages. One disadvantage with stents comprising support members that have a rectangular and square cross-sections is that they have sharp edges, rather than round edges, which can tear the tissue in the lumen. And, possibly, dissect the lumen.

Disadvantages associated with stents comprising support members that have a circular cross-section is that they have an inefficient surface to lumen wall coverage for the mass of material used. Nor are they optimized for radial strength. For example, a Boneau stent is collapsed along a circumferential plane by closing the crowns. And because the material is round, it has the same strength in the circumferential plane as it would if the crown was bent in the other direction. Referring again to FIGS. 2a and 2b, the side portions 24,24′,24″ and 26,26′,26″ lie along a neutral axis which does not need as much material support as the top and bottom portions 20,20′,20″ and 22,22′,22″. In addition, the circular cross-section of such a support member has a large lumen wall stand-off thickness, which decreases the overall inner diameter of the stent.

According to the present invention, the cross-section of conventional stent support members are changed through a swaging technique which changes the profile of the stent such that material from the low stressed locations in the support members are moved to higher stress areas.

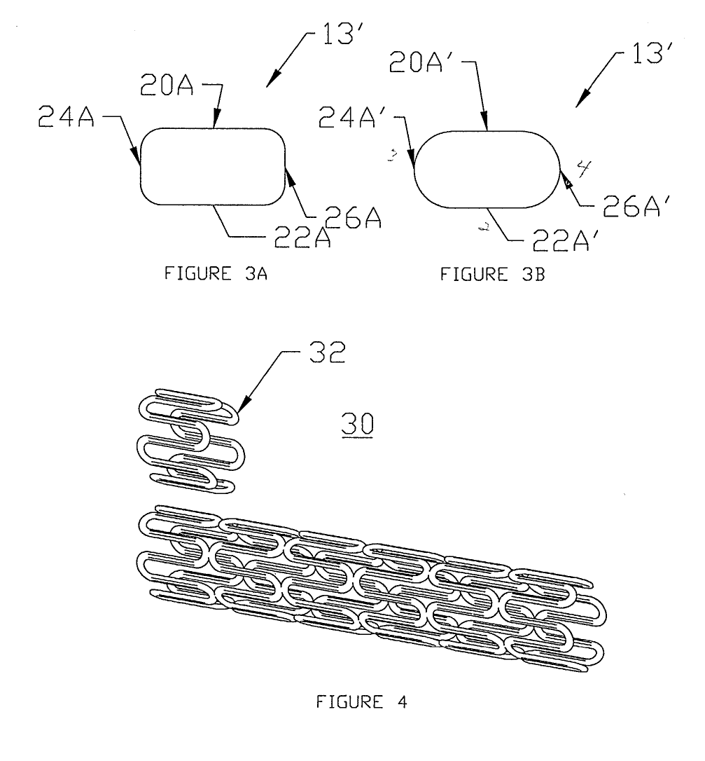

FIGS. 3a and 3b are cross-section views of a profiled member 13,13′,13″ of a stent in accordance with the present invention. FIG. 3a is a cross-section of the section 13 shown in FIG. 2b after profiling, and FIG. 3b a cross-section of the section 13′ shown in FIG. 2b after profiling. The profiling process results in the top and bottom portions being substantially flat and/or with the surfaces of the top and bottom portions exhibiting substantially similar absolute radii of curvature.

As shown, the profiling process has moved material from the neutral axis of the support member, the side portions 24a and 26a, to the more stressed regions of the stent, the top and bottom potions 20a and 22a. As will be appreciated by those of ordinary skill in this art, this process increases the moment of inertia for the stent in the circumferential plane. In addition, the cross-section of a profiled support member has a small lumen wall stand-off thickness, which increases the overall inner diameter of the stent thereby increasing lumen size.

FIG. 4 is an isometric view of a Boneau stent similar to that shown in FIG. 1 which has undergone the process of swaging in accordance with the present invention to produce a profiled stent 30. As shown, each of the sections 32 comprising the stent 30 have two opposing flat sides and rounded edges. The effect of profiling the support members of a stent can also be seen by comparing an end view of a non-profiled stent with the end view of a profiled stent.

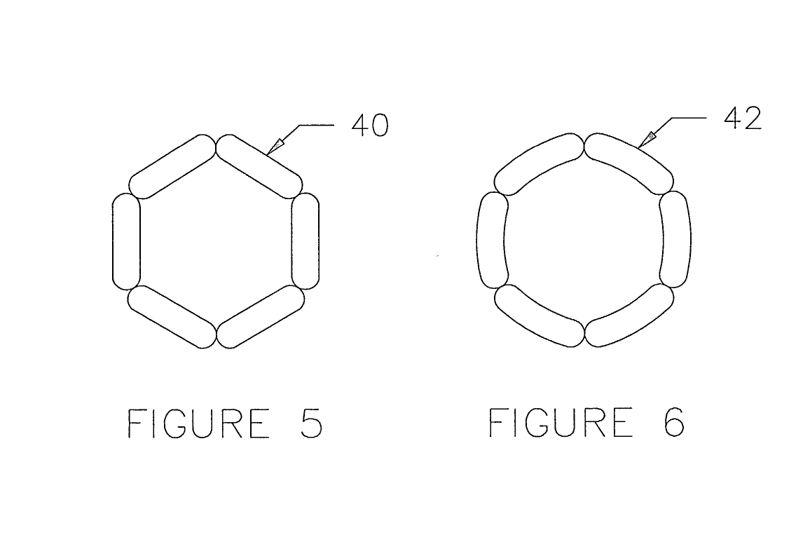

FIG. 5 is an end view of a six crown non-profiled Boneau stent. And FIG. 6 is an end view of a six crown profiled Boneau stent. Both the non-profiled stent 40 and the profiled stent 42 are shown in compressed form and rolled down onto a catheter (not shown); therefore, only the crowns of the stent are visible. Due to the resulting smaller cross-section shape, the crowns of the profiled stent 42 appear longer and narrower than the crowns of the non-profiled stent 40.

Profiling a stent 42 in this manner has many advantages including increasing the radial strength of the stent, and increasing the efficiency of surface coverage. Furthermore, the rounded edges are retained on the stent, which provides less traumatic trackability as the stent is advanced through a lumen. This avoids dissection of the lumen as might occur with tubular-slotted stents. In addition, since the profiled stent contains the same volume of material it maintains its radiopacity or visibility during fluoroscopy.

In a preferred embodiment, the entire stent is profiled. The stent however could be preferentially profiled by profiling only the struts 13 or only the crowns 14 where most of the stress occurs in the instance of a multi-section stent, or by selectively profiling one or more stent sections. Pending application Ser. No. 08/620,878 entitled, “STENTS FOR SUPPORTING LUMENS IN LIVING TISSUE” discloses a strain relief stent in which the end sections of the stent are circumferentially weaker than the middle sections of the stent. The method of profiling a stent in accordance with the present invention may be used to create such a strain relief stent. This may be done by profiling only the middle sections of the stent, leaving the end sections unprofiled. The profiled sections will have a thicker web, causing the material to plastically deform at a lower deflection, since the rounded cross-sections of the non-profiled end-sections will be more flexible and more resilient than the middle sections.

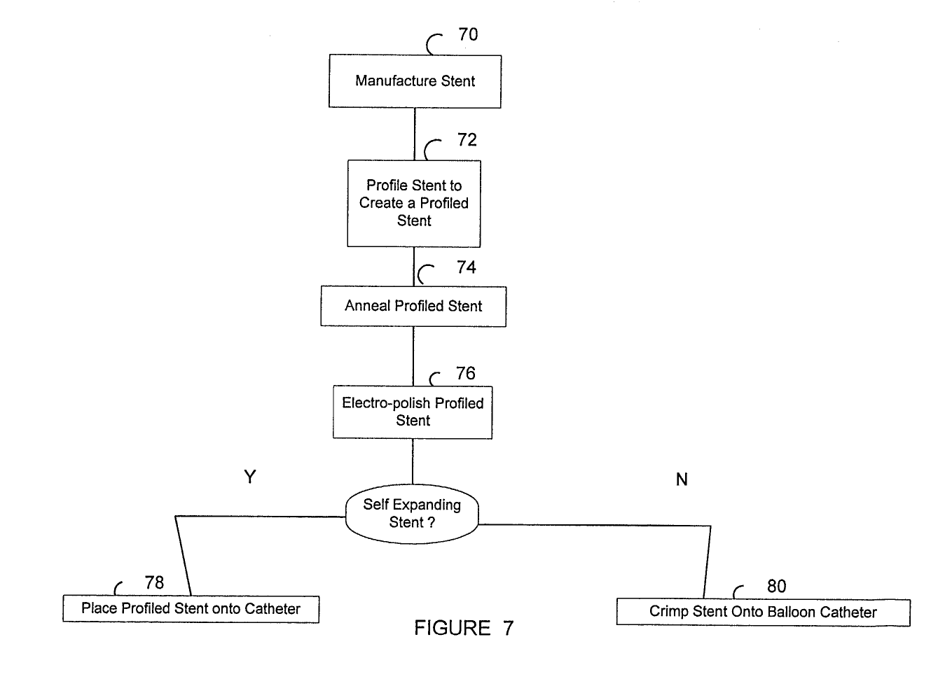

FIG. 7 is a flow chart depicting the process of producing a profiled stent in accordance with the present invention. The process begins by manufacturing a conventional stent in step 70. The particular type of stent manufactured may include self-expandable stents or balloon-expandable stents, and tubular-slotted stents or wire-like stents as described above.

After the stent is manufactured, the stent is swaged in order to calibrate the walls of the stent to a desired thickness in step 72. After swaging, the profiled stent is annealed in step 74 to soften and de-stress the material comprising the stent. After annealing, the stent is electro-polished in step 76.

If the stent is self expanding, then the stent can be placed on a catheter is step 78. If the stent is a balloon inflatable stent, then the stent is crimped onto a balloon catheter in step 80 for subsequent insertion into a lumen.

Many methods for swaging a stent are available. In one preferred embodiment of the present invention, a stent is profiled by swaging the stent by either using a swaging machine or by using a collet. In another embodiment, the stent is profiled using a roller method. In yet another embodiment, the stent is profiled using a sizing tube and forming tool.

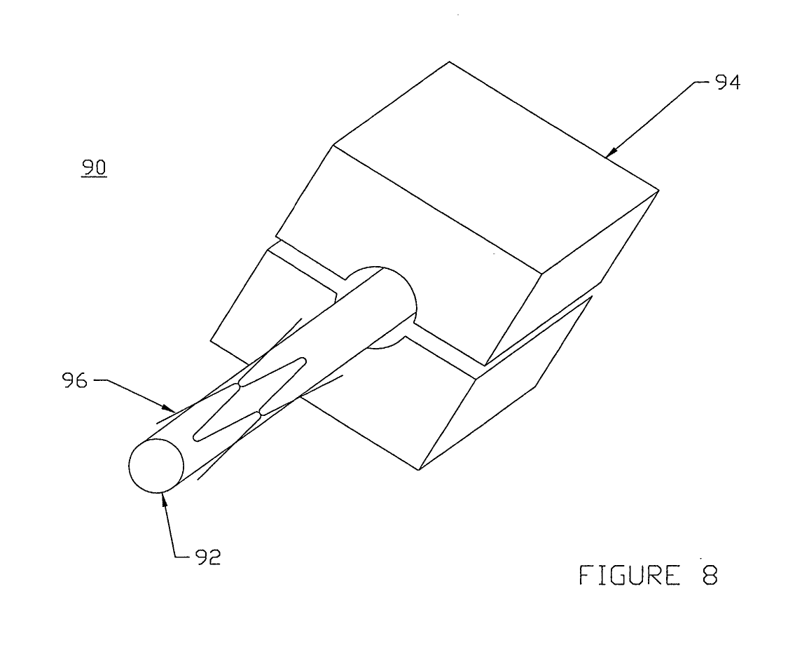

FIG. 8 is a block diagram showing the profiling of a stent using a rotary swaging machine 90. The rotary swaging machine 90 includes a mandrel 92 over which a conventional stent 96 is placed, and a die set 94. The stent is swaged by passing the stent and mandrel 92 through the rotating die set 94 while the die set is repeatedly opened and closed. The closed die forces the stent to conform to the annular space defined between the mandrel and the closed die. This plastically deforms the stent. A non-rotary swage machine is also suitable.

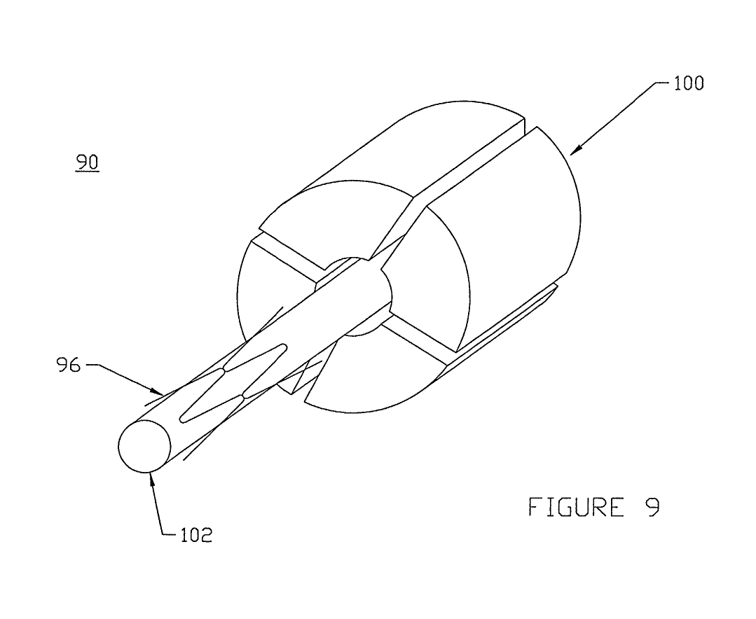

FIG. 9 is a block diagram showing the profiling of stent 96 using a collect 100. Similar to the rotary swage machine 90, a conventional stent is placed over mandrel 102 which is in turn placed into the collet 100. Collet 100 is closed, forcing the stent to conform to the annular space defined between the mandrel 102 and the closed collect 100.

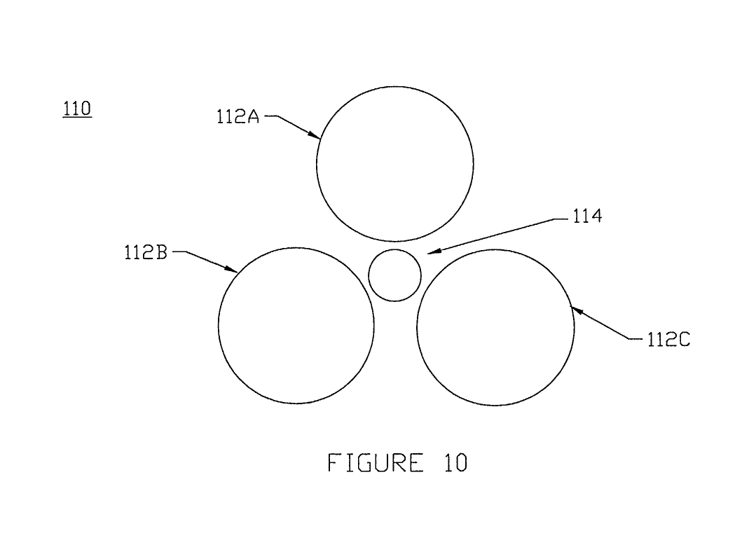

FIG. 10 is a block diagram showing the profiling of a stent using a roller machine 110. The roller machine 110 includes a set of three rollers 112a-112c and a mandrel 114 for supporting the stent. Roller 112a is fed into rollers 112b and 112c, thereby compressing the stent against the mandrel 114. The rollers 112 could also be tapered, where the mandrel 114 and the stent are fed through the tapered rollers 112. The thickness of the resulting profiled stent is controlled by the gap between the rollers 112 and the mandrel 114.

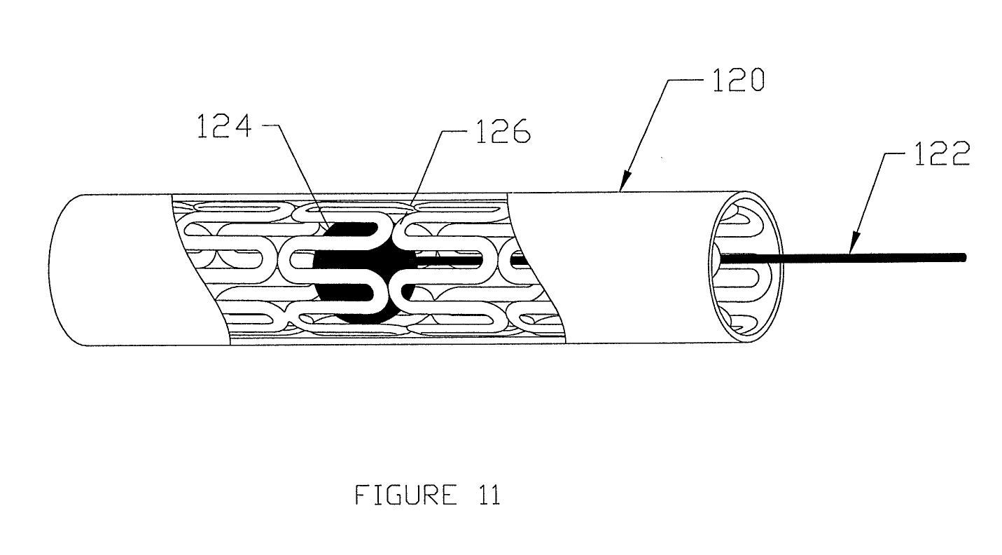

FIG. 11 is a block diagram illustrating the profiling of stent 126 using a sleeve, or sizing tube, 120 and a forming tool 122. Forming tool 122 includes a spherical portion 124 at one end thereof. Stent 126 is inserted into sizing tube 120, and then forming tool 122 is drawn through the interior of stent 126. Rather than deforming the stent material by the use of inwardly directed radial force, as, for example, in the rotary swaging method described above, this process of drawing the forming tool through the sizing tube creates external forces between the stent and the sizing tube, thereby profiling the stent.

A profiled stent and method therefor has been disclosed. Although the present invention has been described in accordance with the embodiments shown, one of ordinary skill in the art will readily recognize that there could be variations to the embodiments and those variations would be within the spirit and scope of the present invention. For example, with respect to tubular-slotted stents, the stent can be cut from a sheet, crushed between a flat plate forming die, and then rolled, with a forming bar or similar tool, and welded. With respect to wire-like stents, after bending the wire into the desired shape it is similarly rolled and the two ends of the wire joined.

Moreover, the instant invention can be used to calibrate the wall thickness of any stent and achieve uniform wall thickness. Additionally, swaging methods of the present invention can be used on any stent material e.g. metal, metal alloy, shape-memory alloy, polymers, etc., that can be plastically deformed. Accordingly, many modifications may be made by one of ordinary skill in the art without departing from the spirit and scope of the appended claims.

What is claimed is:

1. A profiled stent for helping to hold open a lumen in living tissue comprising:
at least one support member having at least a first side, a second side, and a third side, and three rounded edges defined where the first and the second sides, the first and the third sides, and the second and the third sides meet.

2. The stent according to claim 1 further including a fourth side, wherein the second side is positioned opposite the first side, the third side adjoins the first and second sides, and the fourth side is positioned opposite the third side and adjoins the first and second sides, the profiled stent further including four corners defined where the first, second, third, and fourth sides adjoin, wherein the support member is profiled such that each of the four corners are round, while the first and second sides are substantially flat.

3. The stent according to claim 2 wherein the profiled stent includes a plurality of supporting members.

4. The stent according to claim 3 wherein all of the supporting members are profiled.

5. The stent according to claim 2 wherein the first and second sides are flattened by a swaging process.

6. The stent according to claim 2 further comprising:
the stent having a plurality of stent sections;
each of said plurality of stent sections having at least one support member; and
at least one of said plurality of stent sections being profiled.

7. A profiled stent for helping to hold open a lumen comprising:
at least one stent section;
said at least one stent section including at least one supporting member having a first side, a second side positioned opposite the first side, a third side adjoining the first and second sides, a fourth side positioned opposite the third side adjoining the first and second sides, and
four corners defined by the first, second, third, and fourth sides, wherein the four corners of the at least one support member are round, and the first and second sides are profiled such as to exhibit substantially similar radii of curvature.

8. The stent according to claim 7 wherein said first and second sides are profiled to be substantially flat.

9. The stent according to claim 7 wherein said at least one support member is profiled.

10. The stent according to claim 7 wherein said at least one stent section further comprises:
a plurality of support members; and all said support members are profiled such that the surfaces of the first and second sides exhibit substantially similar radii of curvature.

11. The stent according to claim 7 further comprising:
a plurality of stent sections; each of said plurality of stent sections having at least one support member; and
at least one of said stent sections is profiled.

12. A method for producing a stent to be placed in a lumen in living tissue comprising the steps of:
manufacturing the stent; and
profiling the stent to create a profiled stent.

13. The method according to claim 12 wherein the lumen has an inner wall and the manufactured stent comprises at least one stent section including at least one supporting member having an outer wall and an inner surface radially opposing the outer surface, further including the step of:
profiling the manufactured stent such that the at least one support member of the profiled stent has a cross-section that is flatter than the cross-section of the at least one support member of the manufactured stent; and wherein the outer and inner surfaces are profiled to also exhibit substantially similar radii of curvature.

14. The method according to claim 12 further including the step of thermally processing the profiled stent.

15. The stent according to claim 14 wherein the stent is thermally processed by annealing.

16. The method according to claim 12 further including the step of electro-polishing the profiled stent.

17. The method according to claim 12 further including the step of profiling the stent using a swage machine.

18. The method according to claim 17 wherein the swage machine includes a mandrel and a die and further includes the steps of:
loading said stent onto the mandrel; and
drawing said mandrel through the die.

19. The method according to claim 12 further including the steps of:
placing the manufacturing stent into a sleeve; and
advancing a forming tool through said stent in order to profile said stent.
